(19) United States
(12) Patent Application Publication
(10) Pub. No.: US 2024/0156675 A1
(43) Pub. Date: May 16, 2024

(54) SYSTEM AND METHOD FOR APPLYING VIBRATORY STIMULUS IN A WEARABLE DEVICE

(71) Applicant: Encora, Inc., Boston, MA (US)

(72) Inventors: Daniel Carballo, Boston, MA (US); Allison Davanzo, Green Cove Springs, FL (US)

(21) Appl. No.: 18/405,777

(22) Filed: Jan. 5, 2024

Related U.S. Application Data
(63) Continuation of application No. 18/447,656, filed on Aug. 10, 2023.
(60) Provisional application No. 63/371,145, filed on Aug. 11, 2022.

(51) Int. Cl. A61H 23/02

(52) U.S. Cl. CPC A61H 23/02; A61H 2201/1638; A61H 2201/165; A61H 2201/5005; A61H 2201/5007; A61H 2205/06; A61H 2230/605

(57) ABSTRACT

In an embodiment, a wearable device for vibratory stimulation is presented. The wearable device includes a sensor configured to receive data and generate sensor output. The wearable device includes a processor in communication with the sensor and a memory communicatively connected to the processor. The memory includes instructions configuring the processor to receive the sensor output from the sensor. The processor is configured to determine a symptom of a movement disorder of a user based on the sensor output. The processor is configured to calculate a waveform output based on the symptom of the movement disorder. The processor is configured to command a transducer in communication with the processor to apply the waveform output to the user to reduce the symptom of the movement disorder.

### CROSS-REFERENCE TO RELATED APPLICATION

This application is a continuation of U.S. application Ser. No. 18/447,656, filed Aug. 10, 2023, which claims priority to and the benefit of U.S. Provisional Application No. 63/371,145, filed Aug. 11, 2022, and titled “Systems and Methods for Applying Vibratory Stimulus in a Wearable Device”, which is incorporated herein in its entirety.

### TECHNICAL FIELD

This disclosure relates to systems and methods for applying stimulus. In particular, the current disclosure relates to systems and methods for applying stimulus in a wearable device.

### BACKGROUND

There are approximately 10 million people living with movement disorders in the world today. Many modern therapies for movement disorders can be invasive and costly. Modern movement disorder treatments and/or therapies can be improved.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In an embodiment, a wearable device for vibratory stimulation is presented. The wearable device includes a sensor configured to receive data and generate sensor output. The wearable device includes a processor in communication with the sensor and a memory communicatively connected to the processor. The memory includes instructions configuring the processor to receive the sensor output from the sensor. The processor is configured to determine a symptom of a movement disorder of a user based on the sensor output. The processor is configured to calculate a waveform output based on the symptom of the movement disorder. The processor is configured to command a transducer in communication with the processor to apply the waveform output to the user to reduce the symptom of the movement disorder.

In another embodiment, a method of providing vibratory stimulation through a wearable device is presented. The method includes receiving through a sensor of a wearable device data of a user. The method includes generating, through the sensor, sensor output based on the data of the user. The method includes communicating the sensor output to a processor of the wearable device. The method includes determining by the processor a symptom of a movement disorder based on the sensor output. The method includes calculating by the processor a waveform output based on the symptom of the movement disorder. The method includes commanding a transducer in communication with the processor to apply the waveform output to the user.

### BRIEF DESCRIPTION OF THE DRAWINGS

The foregoing aspects and many of the attendant advantages of embodiments of the present disclosure will become more readily appreciated as the same become better understood by reference to the following detailed description, when taken in conjunction with the accompanying drawings.

FIG. 1 illustrates a system for mitigating a movement disorder.

FIG. 2 shows the flexor muscles and tendons of the wrist, fingers, and thumb.

FIG. 3 shows the extensor muscles and tendons of the wrist, fingers, and thumb.

FIG. 4 depicts the somatosensory afferents targeted, which are the subset of cutaneous mechanoreceptors.

FIG. 5 shows the locations of the upper limb dermatomes innervated by the C5, C6, C7, C8, and T1 spinal nerves.

FIG. 6 illustrates a waveform parameter selection process.

FIG. 7 illustrates a user input process for waveform parameter selection.

FIG. 8 is a flow diagram of a method of mitigating a movement disorder.

FIG. 9 is an illustration of a wearable device.

FIG. 10 is an exploded side view of a wearable device.

FIG. 11 illustrates a machine learning module.

FIG. 12 illustrates a block diagram of a computing system that may be implemented with any system, process, or method as described throughout this disclosure.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION OF EXEMPLARY EMBODIMENTS

Aspects of the present disclosure can be used to provide a reduction in movement disorder symptoms through a wearable medical device. In an embodiment, a wearable medical device may provide vibratory stimulus to a body part of a user. Another aspect of the present disclosure can be used to apply stimulation around a circumference of a user's wrist through a wristband which may allow for stimulation of five distinct somatosensory channels via the C5-T1 dermatomes as well as an additional fifteen proprioceptive channels via the tendons passing through the wrist. This may allow for a total of twenty distinct channels with a wristband formfactor which would also be much less cumbersome than an electrical glove.

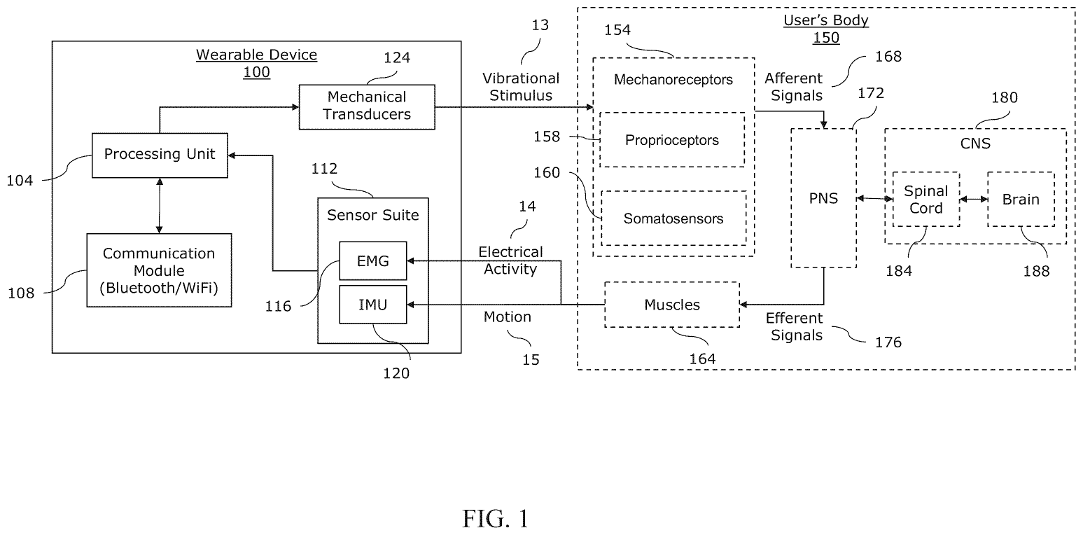

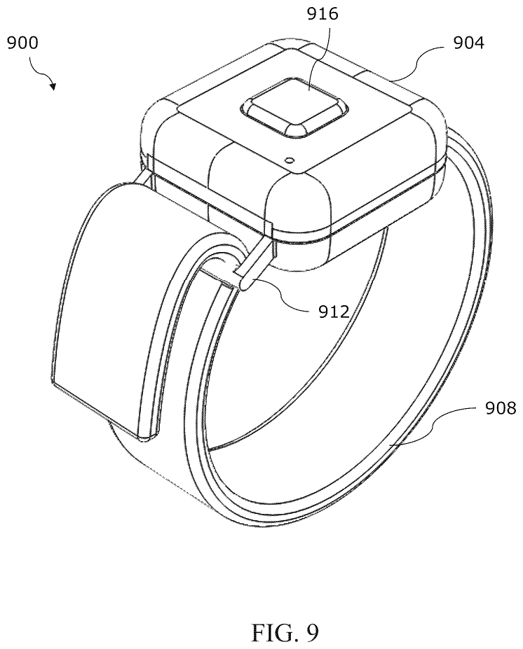

FIG. 1 illustrates a system 100 for mitigating a movement disorder in accordance with an embodiment of the present invention. The system 100 may include a wearable device 100. The wearable device 100 may include a processor, such as processing unit 104, and a memory communicatively connected to the processing unit 104. A memory of the wearable device 100 may contain instructions configuring the processing unit 104 of the wearable device 100 to perform various tasks. The wearable device 100 may include a communication module 108. A “communication module” as used throughout this disclosure is any form of software and/or hardware capable of transmission of electromagnetic energy. For instance, the communication module 108 may be configured to transmit and receive radio signals, Wi-Fi signals, Bluetooth® signals, cellular signals, and the like. The communication module 108 may include a transmitter, receiver, and/or other component. A transmitter of the communication module 108 may include, but is not limited to, an antennae. Antennas of the communication module 108 may include, without limitation, dipole, monopole, array, loop, and/or other antennae types. A receiver of the communication module 108 may include an antenna, such as described previously, without limitation. The communication module 108 may be in communication with the processing unit 104. For instance, the processing unit 104 may be physically connected to the communication module 108 through one or more wires, circuits, and the like. The processing unit 104 may command the communication module 108 to send and/or receive data transmissions to one or more other devices. For instance, and without limitation, the communication module 108 may transmit vibrational stimulus data, motion data of the user's body 150, electrical activity of the user's muscles 164, and the like. In some embodiments, the communication module 108 may transmit treatment data. Treatment data may include, without limitation, symptom severity, symptom type, vibrational stimulus 13 frequency, data from the sensor suite 112, and the like. The communication module 108 may communicate with one or more external computing devices such as, but not limited to, smartphones, tablets, laptops, desktops, servers, cloud-computing devices, and the like. The wearable device 100 may be as described further below with reference to FIG. 9.

With continued reference to FIG. 1, the wearable device 100 may include one or more sensors. A “sensor” as used throughout this disclosure is an element capable of detecting a physical property. Physical properties may include, but are not limited to, kinetics, electricity, magnetism, radiation, thermal energy, and the like. In some embodiments, the wearable device 100 may include a sensor suite 112. A “sensor suite” as used throughout this disclosure is a combination of two or more sensors. The sensor suite 112 may have a plurality of sensors, such as, but not limited to, two or more sensors. The sensor suite 112 may have two or more of a same sensor type. In other embodiments, the sensor suite 112 may have two or more differing sensor types. For instance, the sensor suite 112 may include an electromyography sensor (EMG) 116 and an inertial measurement unit (IMU) 120. The IMU 120 may be configured to detect and/or measure a body's specific force, angular rate, and/or orientation. Other sensors within the sensor suite 112 may include are accelerometers, gyroscopes, impedance sensors, temperature sensors, and/or other sensor types, without limitation. The sensor suite 112 may be in communication with the processing unit 104. A communication between the sensor suite 112 and the processing unit 104 may be an electrical connection in which data may be shared between the sensor suite 112 and the processing unit 104. In some embodiments, the sensor suite 112 may be wirelessly connected to the processing unit 104, such as through, but not limited to, a Wi-Fi, Bluetooth®, or other connection. In some embodiments, one or more components of the wearable device 100 may be the same as described in U.S. application Ser. No. 16/563,087, filed Sep. 6, 2019, and titled “Apparatus and Method for Reduction of Neurological Movement Disorder Symptoms Using Wearable Device”, the entirety of which is incorporated herein by reference.

One or more sensors of the sensor suite 112 may be configured to receive data from a user, such as the user's body 150. Data received by one or more sensors of the sensor suite 112 may include, but is not limited to, motion data, electric data, and the like. Motion data may include, but is not limited to, acceleration, velocity, angular velocity, and/or other types of kinetics. In some embodiments the IMU 120 may be configured to receive motion 15 from the user's body 150. The motion 15 may include, without limitation, vibration, acceleration, muscle contraction, and/or other aspects of motion. The motion 15 may be generated from one or more muscles 164 of the user's body 150. The muscles 164 may include, but are not limited to, wrist muscles, hand muscles, forearm muscles, and the like. In an embodiment, the motion 15 generated from the muscles 164 of the user's body 150 may be involuntarily generated by one or more symptoms of a movement disorder of the user's body 12. A movement disorder may include, without limitation, Parkinson's disease (PD), post stroke recovery, and the like. Symptoms of a movement disorder may include, but are not limited to, stiffness, freezing of gait, tremors, shaking, involuntary muscle contraction, and/or other symptoms. In other embodiments, the motion 15 generated from the muscles 164 of the user's body 150 may be voluntary. For instance, a user may actively control one or more of their muscles 164, which may generate motion 15 that may be detected and/or received by a sensor of the sensor suite 112.

Still referring to FIG. 1, one or more sensors of the sensor suite 112 may be configured to receive electrical data, such as the electrical activity 14 that may be generated by one or more of the muscles 164. Electric data may include, but is not limited to, voltages, impedances, currents, resistances, reactances, waveforms, and the like. For instance, the electrical activity 14 may include an increase in current and/or voltage of one or more of the muscles 164 during a contraction of one or more of the muscles 164. The EMG 116 of the sensor suite 112 may be configured to receive and/or detect the electrical activity 14 generated by the muscles 164. In some embodiments, one or more sensors of the wearable device 11 may be configured to generate sensor output. “Sensor output” as used in this disclosure is information generated by one or more sensing devices. Sensor output may include, but is not limited to, voltages, currents, accelerations, velocities, and/or other output. Sensor output generated from one or more sensors of the sensor suite 112 may be communicated to the processing unit 104, such as through a wired, wireless, or other connection. The processing unit 104 may be configured to determine a symptom of a movement disorder based on sensor output received from one or more sensors. The processing unit 104 may be configured to determine symptoms such as, but not limited to, stiffness, tremors, freezing of gait, and the like. Freezing of gait refers to a symptom of Parkinson's disease in which a person with Parkinson's experiences sudden, temporary episodes of inability to step forward despite an intention to walk. An abnormal gait pattern can range from merely inconvenient to potentially dangerous, as it may increase the risk of falls. Stiffness may refer to a muscle of a person with Parkinson's disease that may contract and become rigid without the person wanting it to. The processing unit 104 may compare one or more values of sensor output from the sensor suite 112 to one or more values associated with one or more symptoms of a movement disorder. For instance, the processing unit 104 may compare sensor output of one or more sensors of the sensor suite 112 to one or more stored values that may already be associated with one or more symptoms of a movement disorder. As a non-limiting example, acceleration of a user's arm of about 1 in/s to about 3 in/s may correspond to a symptom of a light tremor.

In some embodiments, the processing unit 104 may utilize a classifier or other machine learning model that may categorize sensor output to categories of symptoms of a movement disorder. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Processor 104 and/or another device may generate a classifier using a classification algorithm, defined as a process whereby a processor derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, Fisher's linear discriminant, quadratic classifiers, decision trees, boosted trees, random forest classifiers, kernel estimation, learning vector quantization, and/or neural network-based classifiers.

With continued reference to FIG. 1, a classifier may be generated, as a non-limiting example, using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(AB) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table.

The processor 104 may calculate a likelihood table by calculating probabilities of different data entries and classification labels. The processor 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, a classifier may be generated using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample. This may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

A classifier may be trained with training data correlating motion data and/or electric data to symptoms of a movement disorder. Training data may be received through user input, external computing devices, and/or previous iterations of training. As a non-limiting example, the IMU 120 may receive the motion 15 generated by the muscles 164 and may generate sensor output including acceleration values which may be communicated to the processing unit 104. The processing unit 104 may classify and/or categorize the sensor output to a symptom of freezing of gait.

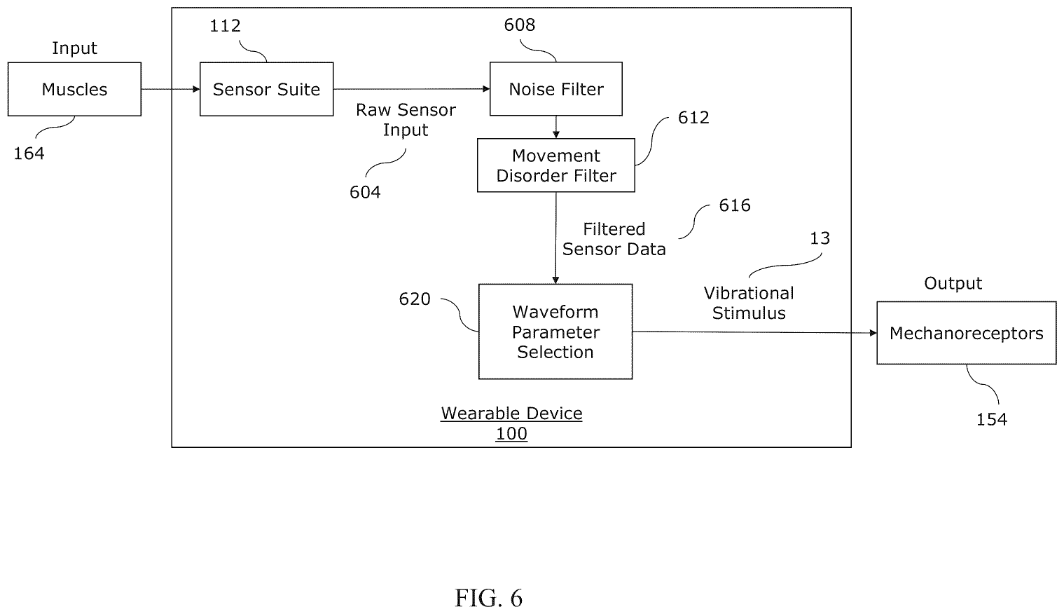

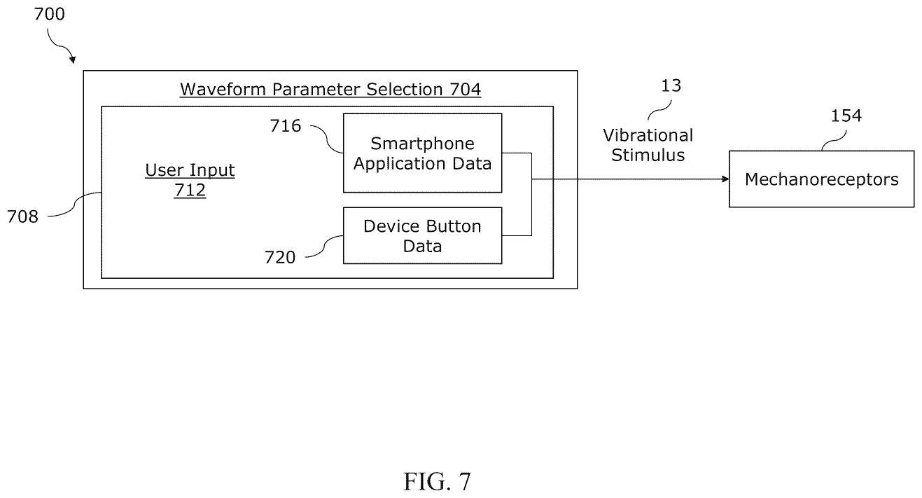

Still referring to FIG. 1, the processing unit 104 may train a classifier with training data correlating motion and/or electrical data to symptoms of a movement disorder. In other embodiments, training of a classifier and/or other machine learning model may occur remote from the processor 104 and the processor 104 may be sent one or more trained models, weights, and the like of a classifier, machine learning model, and the like. Training data may be received by user input, through one or more external computing devices, and/or through previous iterations of processing. A classifier may be configured to input sensor output, such as output of the sensor suite 112, and categorize the output to one or more groups, such as, but not limited to, tremors, stiffness, freezing of gait, and the like. The processing unit 104 may calculate a waveform output based on sensor output generated by one or more sensors of the wearable device 100. A “waveform output” as used in this disclosure is a signal having a frequency. A waveform output may be generated as a vibrational, electrical, audial, and/or other waveform. A waveform output may include one or more parameters such as frequency, phase, amplitude, channel index, and the like. A channel index may include a channel of mechanoreceptors and/or of an actuator to be used. For instance, a channel index may include one or more channels of mechanoreceptors, actuators to stimulate the mechanoreceptors, and/or a combination thereof. The processing unit 104 may select one or more parameters of a waveform output based on received sensor output from one or more sensors of the wearable device 100. In other embodiments, waveform parameters may be selected by the user. As a non-limiting example, a user may select waveform parameters from a predefined list of waveforms using buttons on the wearable device 100. A predefined list of waveforms may include one or more waveforms having various frequencies, amplitudes, and the like, without limitation. A predefined list of waveforms may be generated through previous iterations of waveform generation. In other embodiments, a predefined list of waveforms may be entered by one or more users. In some embodiments, a predefined list of waveforms may include waveforms for specific symptoms, such as, but not limited to, freezing of gait, tremors, stiffness, and the like. In some embodiments, a user may select specific waveform parameters using an external computing device such as, but not limited to, a smartphone, laptop, tablet, desktop, smartwatch, and the like, which may be in communication with the processing unit 104 through the communication module 108. A waveform output generation may be described in further detail below with reference to FIGS. 6-7.

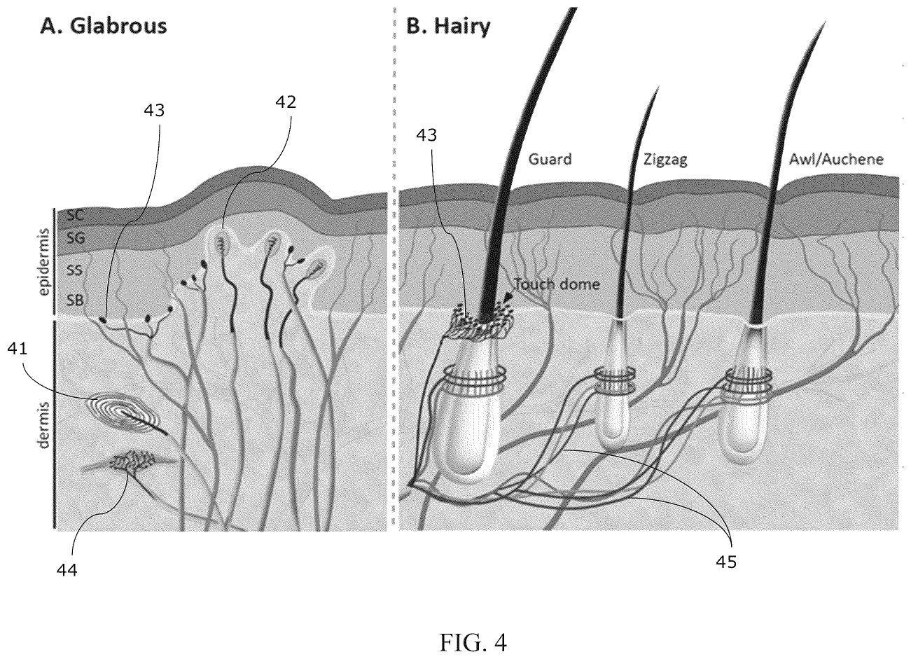

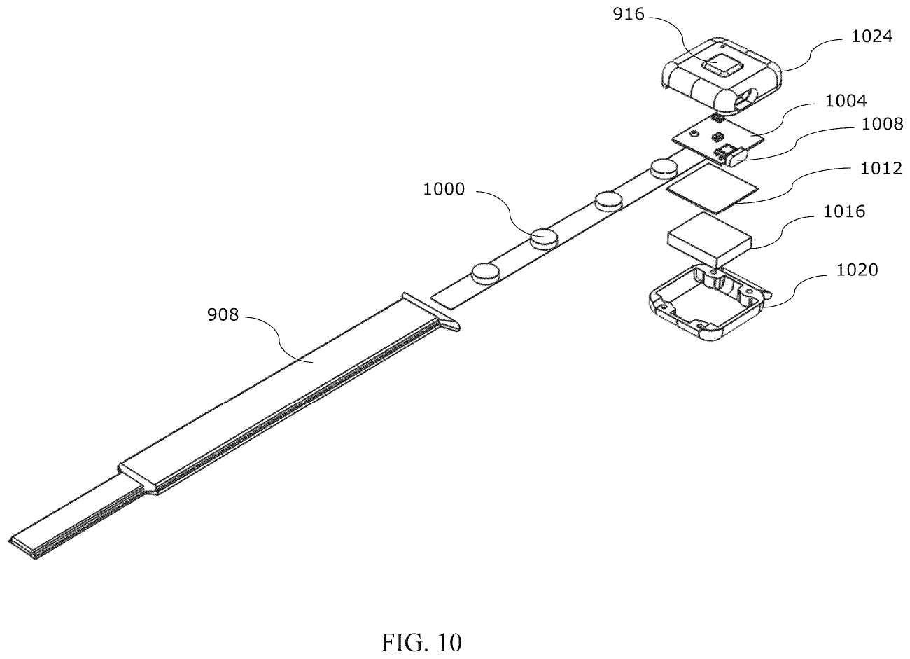

In some embodiments, the processing unit 104 may communicate a waveform output with one or more transducers of the wearable device 100. A “transducer” as used in this disclosure is a device that converts energy from one form to another. For instance, a transducer may include, without limitation, an electric, mechanical, thermal, audial, and/or other types of transducers. The wearable device 100 may include one or more transducers. For instance, the wearable device 100 may include two or more transducers. In some embodiments, the wearable device 100 may include two or more transducers of differing types, such as a mechanical transducer and an electrical transducer, an electrical transducer and an audial transducer, and the like. Transducers of the wearable device 100 may be positioned to provide stimulus, such as through a waveform output, to specific parts of the user's body 150. The wearable device 100 may include one or more mechanical transducers 124 that may be positioned to stimulate one or more mechanoreceptors 154 of the user's body 150. For instance, the mechanical transducers 124 may be positioned along a wristband of the wearable device 100. The wearable device 100 may include, in an embodiment, four mechanical transducers 124 that may be equidistant from one another and positioned within a wristband of the wearable device 100. In other embodiments, the mechanical transducers 124 may be positioned on a surface of a housing of the wearable device 100, as described in further detail below with reference to FIG. 10. The mechanical transducers 124 may include, but are not limited to, piezoelectric motors, electromagnet motors, linear resonant actuators (LRA), eccentric rotating mass motors (ERMs), and the like. The mechanical transducers 124 may be configured to vibrate at up to or more than 200 kHz, in an embodiment. The mechanical transducers 124 may draw energy from one or more batteries from the wearable device 100. For instance, the mechanical transducers 124 may draw about 5 W of power from a battery of the wearable device 100. In some embodiments, the mechanical transducers 124 may have a max current draw of about 90 mA, a current draw of about 68 mA, a 34 mA current draw at 50% duty cycle, and may have a voltage of about 0V to about 5V, without limitation. “Mechanoreceptors” as used throughout this disclosure refer to cells of a human body that respond to mechanical stimuli. The mechanoreceptors 154 may include proprioceptors 158 and/or somatosensors 160. The proprioceptors 158 may include head sems of muscles innervated by the trigeminal nerve. The proprioceptors 158 may be part of one or more areas of a user's limbs, such as, but not limited to, wrists, hands, legs, feet, arms, and the like. The somatosensors 160 may include cells having receptor neurons located in the dorsal root ganglion. The mechanoreceptors 154 may be described in further detail below with reference to FIG. 4.

Still referring to FIG. 1, the processing unit 104 may be configured to command the mechanical transducers 124 to apply the vibrational stimulus 13 to one or more mechanoreceptors 154 of the users body 150. The vibrational stimulus 13 may include a waveform output calculated by the processing unit 104 and applied to the user's body 150 through the mechanical transducers 124. It should be noted that although mechanical transducers 124 are depicted in FIG. 1, other transducers as described above may be used, without limitation. The vibrational stimulus 13 may be applied to the mechanoreceptors 154 through the mechanical transducers 124 which may cause the mechanoreceptors 154 to generate one or more afferent signals 154. An “afferent signal” as used in this disclosure is a neuronal signal in a form of action potentials that are carried toward target neurons. The afferent signals 154 may be communicated to the peripheral nervous system (PNS) 172 of the user's body 150. A “peripheral nervous system” as used in this disclosure is the division of nervous system containing all the nerves that lie outside of the central nervous system. The central nervous system (CNS) 1204 may contain the spinal cord 184 and/or the brain 188 of the user's body 150. The brain 188 may communicate efferent signals 176 to the PNS 172 through the spinal cord 184. “Efferent signals” as used in this disclosure are signals that carry motor information for a muscle to take an action. The efferent signals 176 may include one or more electrical signals that may cause the muscles 164 to contract or otherwise move. For instance, the PNS 172 may input the afferent signals 168 and communicate the afferent signals 168 to the brain 188 through the spinal cord 184. The brain 188 may generate one or more efferent signals 176 and communicate the efferent signals to the PNS 172 through the spinal cord 184. The PNS 172 may communicate the efferent signals 176 to the muscles 164.

The processing unit 104 may act in a closed-loop system. For instance, the processing unit 104 may act in a feedback loop between the data generated from the muscles 164 and the vibrational stimulus 13 generated by the mechanical transducers 124. Further, a closed-loop system may extend through and/or to the PNS 172, CNS 180, brain 188, and the like of the user's body 150 based on the afferent signals 168 and the efferent signals 176. In some embodiments, the processing unit 104 may be configured to act in one or more modes. For instance, the processing unit 104 may act in a first and a second mode. A first mode may include monitor movements of the user's body 150 passively to detect a movement disorder symptom above a threshold. A threshold may include a root mean squared acceleration of 100 mG or 500 mG. A threshold may be set by a user and/or determined through the processing unit 104 based on historical data. Historical data may include sensor and/or waveform output data of a user over a period of time, such as, but not limited to, minutes, hours, weeks, months, years, and the like. A threshold may include, without limitation, one or more acceleration, pressure, current, and/or voltage values. In some embodiments, upon a threshold being reached, the processing unit 104 may be configured to act in a second mode in which the processing unit 104 commands the mechanical transducers 124 to provide the vibrational stimulus 13 to the mechanoreceptors 154.

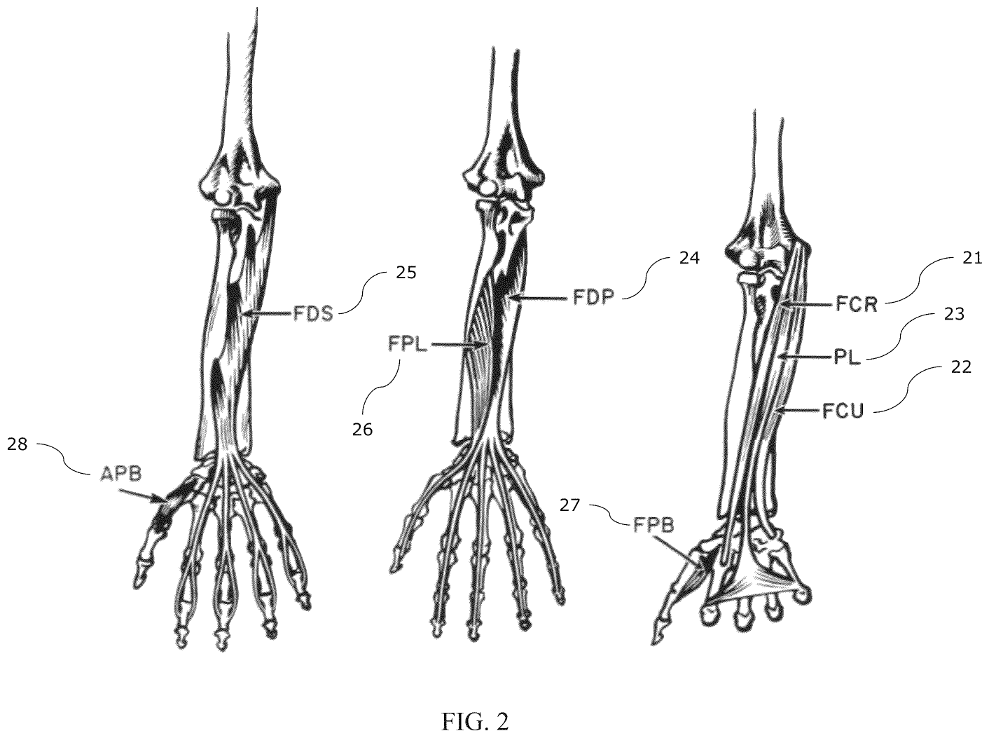

FIG. 2 shows the flexor muscles and tendons of the wrist, fingers, and thumb. The flexors of the wrist are selected from the group consisting of the Flexor Carpi Radialis (FCR) 21, Flexor Carpi Ulnaris (FCU) 22, and the Palmaris Longus (PL) 23. The flexors of the fingers are selected from the group consisting of the Flexor Digitorum Profundus (FDP) 24 and the Flexor Digitorum Superficialis (FDS) 25. The flexors of the thumb are selected from the group consisting of the Flexor Pollicis Longus (FPL) 26, the Flexor Pollicis Brevis (FPB) 27 and the Abductor Pollicis Brevis (APB) 28.

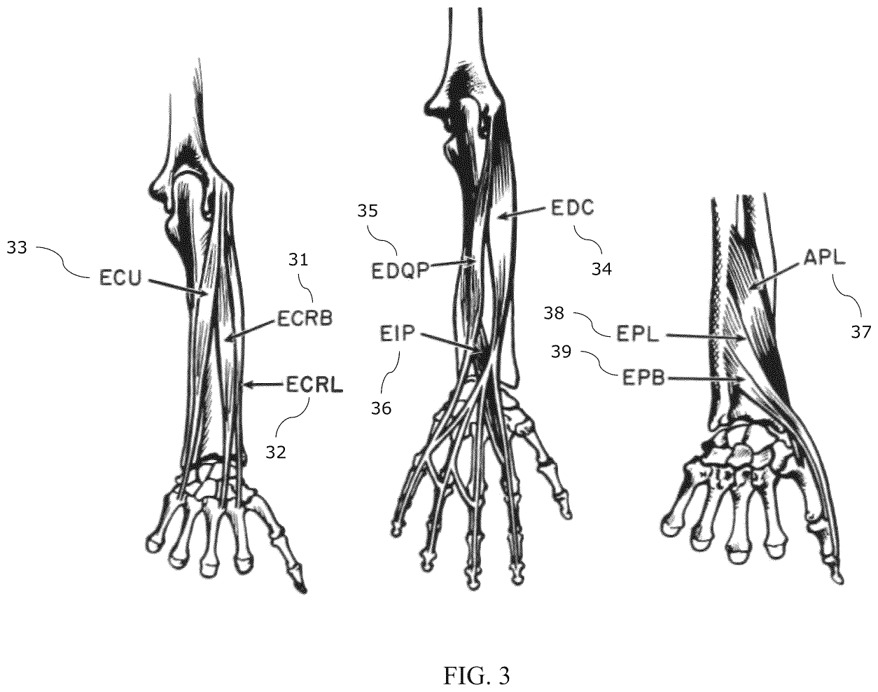

FIG. 3 shows the extensor muscles and tendons of the wrist, fingers, and thumb. The extensors of the wrist are selected from the group consisting of the Extensor Carpi Radialis Brevis (ECRB) 31, Extensor Carpi Radialis Longus (ECRL) 32, and the Extensor Carpi Ulnaris (ECU) 33. The extensors of the fingers are selected from the group consisting of the Extensor Digitorum Communis (EDC) 34, Extensor Digiti Minimi (EDM) or Extensor Digiti Quinti Proprius (EDQP) 35, and the Extensor Indicis Proprius (EIP) 36. The extensors of the thumb are selected from the group consisting of the Abductor Pollicis Longus (APL) 37, Extensor Pollicis Longus (EPL) 38, and the Extensor Pollicis Brevis (EPB) 39.

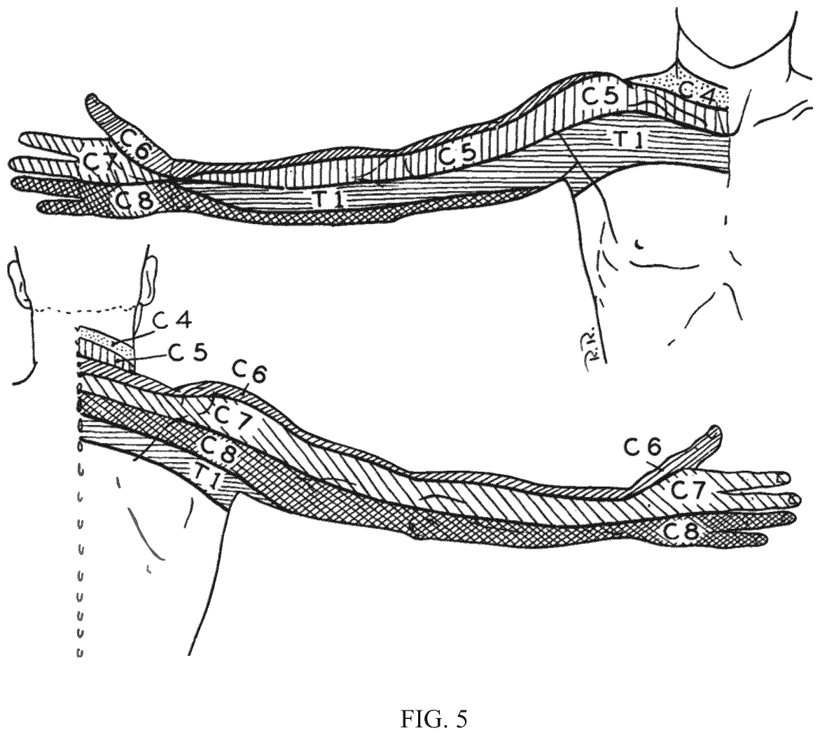

FIG. 4 illustrates various somatosensory afferents that may be targeted. The somatosensory afferents may be a subset of cutaneous mechanoreceptors. The set of cutaneous mechanoreceptors includes the Pacinian corpuscles 41, Meissner corpuscles 42, Merkel complexes 43, Ruffini corpuscles 44, and C-fiber low threshold mechanoreceptors (C-LTMR). The Pacinian corpuscle (PC) 41 is a cutaneous mechanoreceptor that responds primarily to vibratory stimuli in the frequency range of 20-1000 Hz. Meissner corpuscles 42 are most sensitive to low-frequency vibrations between 10 to 50 Hertz and can respond to skin indentations of less than 10 micrometers. Merkel nerve endings 43 are the most sensitive of the four main types of mechanoreceptors to vibrations at low frequencies, around 5 to 15 Hz. Ruffini corpuscles 44 are found in the superficial dermis of both hairy and glabrous skin where they record low-frequency vibration or pressure at 40 Hz and below. C-LTMR 45 are present in 99% of hair follicles and convey input signals from the periphery to the central nervous system. The present invention focuses on the stimulation of cutaneous mechanoreceptors in the upper limb dermatomes innervated by the C5, C6, C7, C8, and T1 spinal nerves, which are depicted in FIG. 5 and labeled according to the corresponding spinal nerve.

Referring now to FIG. 6, a waveform parameter selection system 600 is presented. The system 600 may be local to the wearable device 100, such as by being processed by the processor 104. In other embodiments, the system 600 may be ran through an external computing device, such as, but not limited to, smartphones, tablets, desktops, laptops, servers, cloud-computing devices, and the like. The processing unit 104 may be configured to process raw sensor input 604 received from the sensor suite 112 based on activity of the muscles 164. The raw sensor input 604 may include unprocessed and/or unfiltered sensor data gathered and/or generated by the sensor suite 112. In some embodiments, the processing unit 104 may place the raw sensor input 111 through one or more filters. Filters may include, but are not limited to, noise filters. Filters may include non-linear, linear, time-variant, time-invariant, casual, non-casual, discrete-time, continuous-time, passive, active, infinite impulse response (IIR), finite impulse response (FIR), and the like. The processing unit 104 may use one or more filters to remove noise from the sensor output, such as the noise filter 608. Noise may include unwanted modifications to a signal, such as unrelated sensor output of one or more sensors of the sensor suite 112. The noise filter 608 may use either knowledge of the output waveform to subtract from the sensed waveform or knowledge of the timing of the output waveform to limit sensing to the “off” phases of a pulsing stimulation. In some embodiments, the processing unit 104 may use a filter to remove all information unrelated to a movement disorder, such as through the movement disorder filter 612. Information unrelated to a movement disorder may include specific frequencies and/or ranges of frequencies that may be outside of an indication of a movement disorder. As a non-limiting example, a tremor may have a frequency of about 3 Hz to about 15 Hz, and any frequencies outside of this range may be unrelated to the tremor and subsequently removed through one or more filters. As another non-limiting example, classical rest remor, isolated postural tremor, and kinetic tremor during slow movement may be about 3 Hz to about 7 Hz, 4 Hz to about 9 Hz, and 7 Hz to about 12 Hz, respectively. The processing unit 104 may be configured to filter any frequencies outside of any of the ranges described above. In some embodiments, the processing unit 104 may be configured to extract a fundamental tremor frequency through spectral analysis. A fundamental tremor frequency may be used in one or more filters as a digital bandpass filter with a cutoff frequencies about and below the fundamental frequency. The processing unit 104 may be configured to implement and/or generate one or more filters based on a patient's specific fundamental tremor frequency. The movement disorder filter 612 may be any filter type. In some embodiments, the movement disorder filter 612 may include a 0-15 Hz bandpass filter configured to eliminate any other signal components not caused by a movement disorder. In other embodiments, the movement disorder filter 612 may include a bandpass filter with an upper limit greater than 15 Hz, without limitation. The processing unit 104 may use the movement disorder filter 612 to determine extraneous movement of a user by removing noise unrelated to an extraneous movement of the user. The processing unit 104 may utilize three or more filters, in an embodiment. The processing unit 104 may first use the noise filter 608 to remove noise from the raw sensor input 604 and subsequently use a second filter, such as the movement disorder filter 612, to remove all information unrelated to a movement disorder. In some embodiments, after processing sensor output through one or more filters, filtered sensor data 616 may be generated. In some embodiments, one or more features may be extracted from the filtered sensor data 616. Extraction may include retrieving temporal, spectral, or other features of the filtered sensor data 616. Temporal features may include, but are not limited to, minimum value, the maximum value, first three standard deviation values, signal energy, root mean squared (RMS) amplitude, zero crossing rate, principal component analysis (PCA), kernel or wavelet convolution, or auto-convolution. Spectral features may include, but are not limited to, the Fourier Transform, fundamental frequency, (Mel-frequency) Cepstral coefficients, the spectral centroid, and bandwidth. The processing unit 104 may input the filtered sensor data 616 and/or extraction features of the filtered sensor data 616 into the waveform parameter algorithm 115.

The waveform parameter selection 620 may be a parameter selection algorithm. A parameter selection algorithm may include an algorithm that determines one or more parameters of an output. The waveform parameter selection 620 may include, without limitation, a classification algorithm such as a logistic regression, naïve bayes, decision tree, support vector machine, neural network, random forest, and/or other algorithm. In some embodiments, the waveform parameter selection 620 may be an argmax(FFT) algorithm. The waveform parameter selection 620 may include a calculation of a mean, median, interquartile range, Xth percentile signal frequency, root mean square amplitude, power, log(power), and/or linear or non-linear combination thereof. For instance, and without limitation, the waveform parameter selection 620 may modify a frequency, amplitude, peak-to-peak value, and the like of one or more waveforms. The waveform parameter algorithm 620 that may modify one or more parameters of a waveform output applied to the mechanoreceptors 154, such as the vibrational stimulus 13. In some embodiments, the waveform parameter algorithm 620 may be configured and/or programmed to determine a set of waveform parameters based on a current set of waveform parameters and/or the filtered sensor data 616. As a non-limiting example, the filtered sensor data 616 may include an amplitude of a tremor. The waveform parameter algorithm 620 may compare the tremor amplitude with a current set of waveform parameters to a tremor amplitude observed with a previous set of waveform parameters to determine which of the two sets of waveform parameters results in a lowest tremor amplitude. The set with a lowest resulting tremor amplitude may be used as a baseline for a next iteration of the waveform parameter selection 620, which may compare this baseline to a new set of waveform parameters. The waveform parameter selection 620 may utilize one or more of a Q-learning model, one or more neural networks, genetic algorithms, differential dynamic programming, iterative quadratic regulator, and/or guided policy search. The waveform parameter selection 620 may determine one or more new waveform parameters from a current set of applied waveform parameters based on an optimization model to best minimize a symptom severity of a user. An optimization model may include, but is not limited to, discrete optimization, continuous optimization, and the like. For instance, the waveform parameter selection 620 may utilize an optimization model that may be configured to input the filtered sensor data 616 and/or current waveform parameters of the vibrational stimulus 13 and output a new selection of waveform parameters that may minimize symptom severity of a user. Symptom severity may include, but is not limited to, freezing of gait, stiffness, tremors, and the like.

In some embodiments, the vibratory stimulation 13 may target afferent nerves chosen from the set consisting of the somatosensory cutaneous afferents of the C5-T1 dermatomes and the proprioceptive afferents of the muscles and tendons of the wrist, fingers, and thumb, without limitation. In an embodiment, the vibratory stimulation 13 may be applied around a circumference of a user's wrist which may allow for stimulation of five distinct somatosensory channels via the C5-T1 dermatomes as well as an additional fifteen proprioceptive channels via the tendons passing through the wrist, which may allow for a total of twenty distinct channels. The waveform parameter selection 620 may be configured to generate one or more waveform parameters specific to one or more proprioceptive and/or somatosensory channels. For instance, and without limitation, the waveform parameter selection 620 may select a single proprioceptive channel through a C5 dermatome to apply the vibrational stimulus 13 to. In another instance, and without limitation, the waveform parameter selection 620 may select a combination of a C5 dermatome and T1 dermatome channel. In some embodiments, the waveform parameter selection 620 may be configured to generate a multichannel waveform by generating one or more waveform parameters for one or more proprioceptive and/or somatosensory channels. Channels of a multichannel waveform may be specific to one or more proprioceptive and/or somatosensory channels. In some embodiments, each transducer of a plurality of transducers may each generate a waveform output for a specific proprioceptive and/or somatosensory channel, where each channel may differ from one another, be the same, or a combination thereof. The waveform parameter selection 620 may select any combination of proprioceptive and/or somatosensory channels, without limitation. The waveform parameter selection 620 may select one or more proprioceptive channels to target based on one or more symptoms of a movement disorder. For instance and without limitation, the waveform parameter selection 620 may select both a T1 and C5 channel for stimulation based on a symptom of muscle stiffness. In some embodiments, the waveform parameter selection 620 may include a stimulation machine learning model. A stimulation machine learning model may include any machine learning model as described throughout this disclosure, without limitation. In some embodiments, a stimulation machine learning model may be trained with training data correlating sensor data and/or waveform parameters to optimal waveform parameters. Training data may be received through user input, external computing devices, and/or previous iterations of processing. A stimulation machine learning model may be configured to input the filtered sensor data 616 and/or a current set of waveform parameters and output a new set of waveform parameters. A stimulation machine learning model may be configured to output specific targets for vibrational stimulus, such as one or more proprioceptive and/or somatosensory channels as described above, without limitation. As a non-limiting example, a stimulation machine learning model may input the filtered sensor data 616 and output a set of waveform parameters specific to a C6 and C8 proprioceptive channel. The vibrational stimulus 13 may be applied to one or more mechanoreceptors 154. In some embodiments, where the process 600 happens externally to the wearable device 100, a computing device running the process 600 may communicate one or more waveform parameters to the wearable device 100.

The waveform parameter selection 620 may generate a train of waveform outputs. A train of waveform outputs may include two or more waveform outputs that may be applied to a user sequentially. Periods of time between two or more waveform outputs of a train of waveform outputs may be, without limitation, milliseconds, seconds, minutes, and the like. Each waveform output of a train of waveform outputs may have varying parameters, such as, but not limited to, amplitudes, frequencies, peak-to-peak values, and the like. In some embodiments, a train of waveform outputs may include a plurality of waveform outputs with each waveform output having a higher frequency than a previous waveform output. In some embodiments, each waveform output may have a lower or same frequency than a previous waveform output. The waveform parameter selection 620 may provide a train of waveform outputs until a waveform output reaches a frequency that results in a suppressed output of extraneous movement of a user.

Still referring to FIG. 6, the wearable device 100 may be configured to act in one or more settings. Settings of the wearable device 100 may include one or more modes of operation. A user may be configured to select one or more settings of the wearable device 100 through interactive elements, such as buttons, touch screens, and the like, and/or through a remote computing device, such a through an application, without imitation. Interactive elements and applications may be described in further detail below with reference to FIG. 7.

Settings of the wearable device 100 may include an automatic setting, a tremor reduction setting, a freezing of gait setting, a stiffness setting, and/or an adaptive mode setting.

An automatic setting of the wearable device 100 may include the processing unit 104 automatically selecting a best waveform output based on data generated from one or more sensors of the sensor suite 112. For instance, the waveform parameter selection 620 may select one or more waveform parameters that are generally best suited for current sensor data, such as filtered sensor data 616. An automatic mode of the wearable device 100 may be based on a plurality of data generated from a plurality of users using the wearable device 100 to find one or more averages, standard deviations, and the like, of therapeutic vibrational stimulus 13. In some embodiments, generating an automatic mode of the wearable device 100 may include crowd-sourcing from one or more users. A cloud-computing system may be implemented to gather data of one or more users.

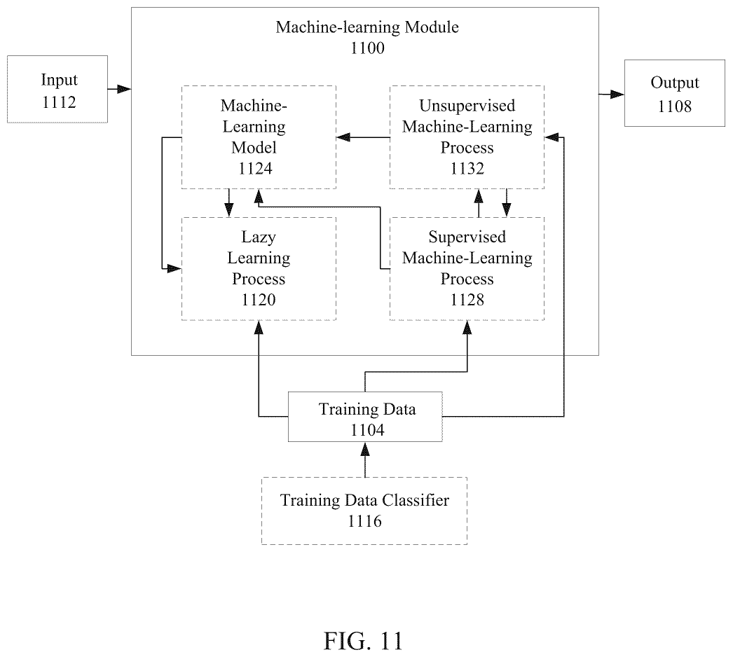

Still referring to FIG. 6, the wearable device 100 may be configured to act in a tremor reduction setting. A tremor reduction setting may include the waveform parameter selection 620 giving more weight or value to filtered sensor data 616 that corresponds to tremors, while lessening weights or values of other symptoms. The waveform parameter selection 620 may be configured to generate one or more waveform parameters that optimize a tremor reduction of a tremor of a user. Optimizing a tremor reduction of a user may include minimizing weights, values, and/or waveform parameters for other symptoms, such as freezing of gait, stiffness, and the like. Likewise, a freezing of gait setting may optimize a reduction in a freezing of gait of a user, a stiffness setting may optimize a reduction in stiffness of a user, and the like. Each setting may be iteratively updated based on data received from crowd-sourcing, user historical data, and the like. For instance, each setting may be continually update to optimize a reduction of symptoms of most users from a population of a plurality of users. In some embodiments, a setting of the wearable device 100 may include an adaptive mode. An adaptive mode may include the waveform parameter selection 620 continually looking for a highest weight of sensor data 616 and/or most severe symptom and generating one or more waveform parameters to reduce said symptom and/or weight. An adaptive mode of the wearable device 100 may utilize a machine learning model, such as described below with reference to FIG. 11. An adaptive mode machine learning model may be trained with training data correlating sensor data and/or weights of sensor data to one or more waveform parameters. Training data may be received through user input, external computing devices, and/or previous iterations of processing. An adaptive mode machine learning model may be configured to input the filtered sensor data 616 and one or more optimal waveform parameters 620 to reduce a symptom having a highest severity. In some embodiments, an adaptive mode machine learning model may be trained remotely and weights of the trained model may be communicated to the wearable device 100 which may reduce processing load of the wearable device 100.

FIG. 7 illustrates a process of waveform parameter selection through a mobile device. The process 700 may be performed by a processor, such as processing unit 104, as described above with reference to FIG. 1, without limitation. The process 700 may include a waveform parameter selection 704. The waveform parameter selection 704 may be the same as the waveform parameter selection 620 as described above with reference to FIG. 6. In some embodiments, an application 708 may be configured to run. The application 708 may be run on, but not limited to, laptops, desktops, tablets, smartphones, and the like. In some embodiments, the application 708 may take the form of a web application. The application 708 may be configured to display data to a user through a graphical user interface (GUI). A GUI may include one or more textual, pictorial, or other icons. A GUI generate by the application 708 may include one or more windows that may display data, such as images, text, and the like. A GUI generated by the application 708 may be configured to display sensor data, stimulation data, and the like. In some embodiments, a GUI generated by the application 708 may be configured to receiver user input 712. User input 712 may include, but is not limited to, keystrokes, mouse input, touch input, and the like. For instance, and without limitation, a user may click on an icon of a GUI generated by the application 708 that may trigger an event handler of the application 708 to perform one or more actions, such as, but not limited to, displaying data through a window, communicating data to another device, and the like. In some embodiments, user input 712 received through the application 708 may generate smartphone application data 716. The smartphone application data 716 may include one or more selections of one or more waveform parameters. Waveform parameters may include, without limitation, amplitude frequency, and the like. Waveform parameters may be as described above with reference to FIGS. 1 and 6. As a non-limiting example, the smartphone application data 716 may include a selection of a higher frequency of a waveform output, the selection being generated by user input through the application 708.

Additionally, and/or alternatively, a user may generate user input 712 through one or more interactive elements of a wearable device. A wearable device may be as described above, without limitation, in FIG. 1. A wearable device may include one or more interactive elements such as, but not limited to, knobs, switches, buttons, sliders, and the like. Each interactive element of a wearable device may correspond to a function. For instance, a button of a wearable device may correspond to an increasing of a frequency of a waveform output while another button of the wearable device may correspond to a decreasing of a frequency of a waveform output. A user may generate device button data 720 through user input 712 of a wearable device. In some embodiments, a wearable device may include a touchscreen or other interactive display through which a user may generate device button data 720 from. In an embodiment, a wearable device may be configured to run the application 708 locally and receive the smartphone application data 716 through a touch screen or other input device that may be part of the wearable device. The waveform parameter selection 704 may be run locally on a wearable device and/or offloaded to one or more computing devices. In some embodiments, the waveform parameter selection 704 may be configured to receive the smartphone application data 716 and/or the device button data 720. The waveform parameter selection 704 may be configured to generate a waveform output, such as the vibrational stimulus 13, based on the smartphone application data 716 and/or the device button data 720. A user may adjust the vibrational stimulus 13 through generating the smartphone application data 716 and/or the device button data 720. The vibrational stimulus 13 may be communicated to one or more mechanoreceptors 154 through one or more transducers, as described above with reference to FIG. 1 and FIG. 6, without limitation.

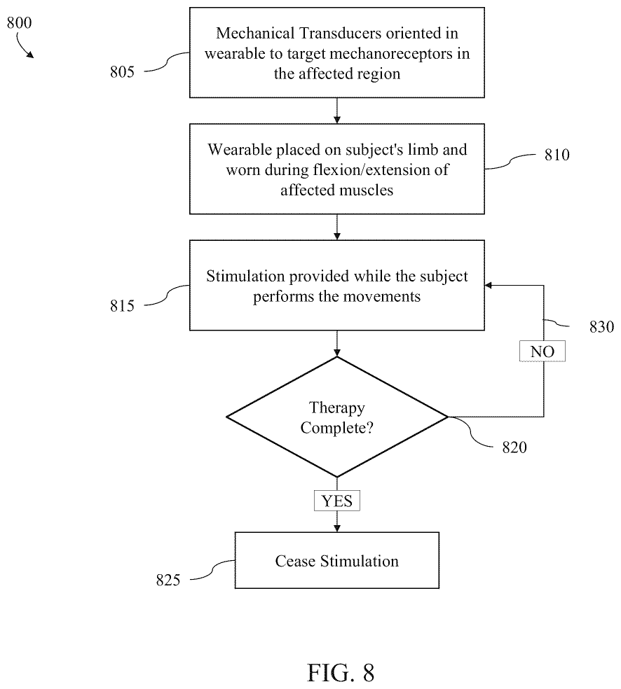

Referring now to FIG. 8, an example of a method of reducing symptoms of a movement disorder 800 is shown. Method 800 be applied to and/or implemented in any process as described. Method 800 may be based on Hebbian learning. Hebbian learning refers to the neuropsychological theory that an increase in synaptic efficacy arises from a presynaptic cell's repeated and persistent stimulation of a postsynaptic cell. For instance, in some embodiments, a user may perform a set of predefined movements during a stimulation, such as the stimulation described above with reference to FIGS. 1 and 5. Performing a predefined set of movements during stimulation may induce neuroplastic changes that may remain after a cessation of stimulation. These movements can be done under the guidance of a physical therapist, occupational therapist, other caretaker, or on one's own. By stimulating the neuronal pathways during movement, the shared synapses between the neurons associated with that movement may be reinforced over time, allowing the therapeutic benefit to persist in the absence of stimulation.

At step 805, the method includes orienting mechanical transducers in a wearable device to target mechanoreceptors in an affected region. For instance, one or more mechanical transducers of a wearable device may be oriented around a user's wrist, arm, leg, and the like.

At step 810, the wearable device may be placed on a subject's limb. The wearable device may be worn during a flexion and/or extension of one or more affect muscles of the user. In some embodiments, the user may perform one or more pre-defined movements such as, but not limited to, walking, making a fist, writing, raising an arm, and the like.

At step 815, stimulation may be provided to the user through the wearable medical device during a movement of the user. For instance, the user may be performing one or more pre-defined movements as described in step 810 and the wearable device may simultaneously stimulate a portion of the user's body. The mechanical transducers supply a vibrational stimulus with a frequency between 1 Hz and 300 Hz.

At step 820, a determination of a completeness of the therapy is made. The determination may be made by a user, professional, application, timer, and/or a combination thereof. In some embodiments, the wearable device may be configured to apply stimulation for a pre-determined amount of time. The pre-determined amount of time may be user selected, professional selected, and/or calculated through historical data by the wearable device. If at step 820, the therapy is deemed complete, the method proceeds to step 825 at which stimulation is ceased. If the therapy is deemed not completed at step 820, the method loops 830 back to step 815 to provide stimulation to the subject through the wearable device. Any one of the steps of method 800 may be implemented as described above with reference to FIGS. 1-7, without limitation.

Referring now to FIG. 9, an illustration of a wearable device 900 is presented. In some embodiments, the wearable device 900 may include a housing 904 that may be configured to house one or more components of the wearable device 900. For instance, the housing 904 of the wearable device 900 may include a circular, ovular, rectangular, square, or other shaped material. In some embodiments, the housing 904 may have a length of about 5 inches, a length of about 5 inches, and a width of about 5 inches, without limitation. In some embodiments, the housing 904 may have a length of about 1.5 inches, a width of about 1.5 inches and a height of about 0.5 inches. The housing 904 of the wearable device 900 may have an interior and an exterior. An interior of the housing 904 of the wearable device 100 may include, but is not limited to, one or more sensors, transducers, energy sources, processors, memories, and the like, such as those described above with reference to FIG. 1. In some embodiments, an exterior of the housing 904 of the wearable device 900 may include one or more interactive elements 916. An “interactive element” as used in this disclosure is a component that is configured to be responsive to user input. The interactive element 916 may include, but is not limited to, buttons, switches, and the like. In some embodiments the wearable device 900 may have a singular interactive element 916. In other embodiments, the wearable device 900 may have two or more interactive elements 916. In embodiments where the wearable device 900 has a plurality of interactive elements 916, each interactive element 916 may correspond to a different function. For instance, a first interactive element 916 may correspond to a power function, a second interactive element 916 may correspond to a waveform adjustment, a third interactive element 916 may correspond to a mode of the wearable device 900, and the like. In some embodiments, the wearable device 900 may include a touch screen display.

In some embodiments, the wearable device 900 may include one or more batteries. For instance, and without limitation, the wearable device 900 may include one or more replaceable batteries, such as lead-acid, nickel-cadmium, nickel-metal hydride, lithium-ion, and/or other battery types. The housing 904 of the wearable device 900 may include a charging port that may allow access to a rechargeable battery of the wearable device 900. For instance and without limitation, the wearable device 900 may include one or more rechargeable lithium-ion battery and a charging port of the housing 904 of the wearable device 900 may be a USB-C, micro-USB, and/or other type of port. A battery of the wearable device 900 may be configured to charge at a rate of about 10 W/hr. A battery of the wearable device 900 may be configured to charge at about 3.7V with a current draw of about 630 mA. A battery of the wearable device 900 may have a capacity of about 2.5 Wh, greater than 2.5 Wh, or less than 2.5 Wh, without limitation. In some embodiments, the wearable device 900 may include one or more wireless charging circuits that may be configured to receive power via electromagnetic waves. The wearable device 900 may be configured to be charged wirelessly at a rate of about 5 W/hr through a charging pad or other wireless power transmission system. In some embodiments, a battery of the wearable device 900 may be configured to be charged at about 460 mA, greater than 460 mA, or less than 460 mA.

Still referring to FIG. 9, the wearable device 900 may include an attachment system. An attachment system may include any component configured to secure two or more elements together. For instance, and without limitation, the wearable device 900 may include a wristband 908. The wristband 908 may include one or more layers of a material. For instance and without limitation, the wristband 908 may include multiple layers of a polymer, such as rubber. The wristband 908 may have an interior and an exterior. An interior and an exterior of the wristband 908 may be a same material, texture, and the like. In other embodiments, an interior of the wristband 908 may be softer and/or smoother than an exterior of the wristband 908. As a non-limiting example, an interior of the wristband 908 may be a smooth rubber material while an exterior of the wristband 908 may be a Velcro material. The wristband 908 may have a thickness of about 2 mm. In other embodiments, the wristband 908 may have a thickness of greater than or less than about 2 mm. The wristband 908 may be a rubber band, Velcro strap, and the like. In some embodiments, the wristband 908 may be adjustable. For instance, the wristband 908 may be a flexible loop that may self-attach through a Velcro attachment system. In some embodiments, the wristband 908 may attach to one or more hooks 912 of an exterior of the housing 904 of the wearable device 900. In some embodiments, the wristband 908 may be magnetic. In other embodiments, the wristband 908 may include a column, grid, or other arrangement of holes that may receive a latching from the hook 912.

Referring now to FIG. 10, an exploded side view of the wearable device 900 is shown. The wearable device 900 may include mechanical transducers 1000. The mechanical transducers 1000 may be housed within the wristband 908. The wristband 908 may be configured to interface with a user's writs. The wearable device 900 may have a top half of a housing 1024 and a bottom half of a housing 1020. In some embodiments, between the top half 1024 and the bottom half 1020, a printed circuit board 1004 (PCB) may be positioned. Further, a silicone square may be positioned to insulate a bottom of the PCB 43, which may be positioned above a battery 1016. The battery 1016 may include protection circuitry to protect from overcharging and unwanted discharging. In some embodiments, the wearable device 900 may include a magnetic connector 1008. The magnetic connector 1008 may be configured to align the wearable device 900 with a charging pad, station, and the like. The magnetic connector 1008 may be configured to receive power wirelessly to recharge the battery 1016. The magnetic connector 1008 may be coupled to the battery 1016 and mounted in the housing 1020 and/or 1024. In some embodiments, the magnetic connector 1008 may be inserted into the PCB 1004. The magnetic connector 1008 may be configured to mate with a connector from an external charger.

Referring to FIG. 11, an exemplary machine-learning module 1100 may perform machine-learning process(es) and may be configured to perform various determinations, calculations, processes and the like as described in this disclosure using a machine-learning process.

Still referring to FIG. 11, machine learning module 1100 may utilize training data 1104. For instance, and without limitation, training data 1104 may include a plurality of data entries, each entry representing a set of data elements that were recorded, received, and/or generated together. Training data 1104 may include data elements that may be correlated by shared existence in a given data entry, by proximity in a given data entry, or the like. Multiple data entries in training data 1104 may demonstrate one or more trends in correlations between categories of data elements. For instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 1104 according to various correlations. Correlations may indicate causative and/or predictive links between categories of data elements, which may be modeled as relationships such as mathematical relationships by machine-learning processes as described in further detail below. Training data 1104 may be formatted and/or organized by categories of data elements. Training data 1104 may, for instance, be organized by associating data elements with one or more descriptors corresponding to categories of data elements. As a non-limiting example, training data 1104 may include data entered in standardized forms by one or more individuals, such that entry of a given data element in a given field in a form may be mapped to one or more descriptors of categories. Elements in training data 1104 may be linked to descriptors of categories by tags, tokens, or other data elements. Training data 1104 may be provided in fixed-length formats, formats linking positions of data to categories such as comma-separated value (CSV) formats and/or self-describing formats. Self-describing formats may include, without limitation, extensible markup language (XML), JavaScript Object Notation (JSON), or the like, which may enable processes or devices to detect categories of data.

With continued reference to refer to FIG. 11, training data 1104 may include one or more elements that are not categorized. Uncategorized data of training data 1104 may include data that may not be formatted or containing descriptors for some elements of data. In some embodiments, machine-learning algorithms and/or other processes may sort training data 1104 according to one or more categorizations. Machine-learning algorithms may sort training data 1104 using, for instance, natural language processing algorithms, tokenization, detection of correlated values in raw data and the like. In some embodiments, categories of training data 1104 may be generated using correlation and/or other processing algorithms. As a non-limiting example, in a body of text, phrases making up a number “n” of compound words, such as nouns modified by other nouns, may be identified according to a statistically significant prevalence of n-grams containing such words in a particular order. For instance, an n-gram may be categorized as an element of language such as a “word” to be tracked similarly to single words, which may generate a new category as a result of statistical analysis. In a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 1104 to be made applicable for two or more distinct machine-learning algorithms as described in further detail below. Training data 1104 used by machine-learning module 1100 may correlate any input data as described in this disclosure to any output data as described in this disclosure, without limitation.

Further referring to FIG. 11, training data 1104 may be filtered, sorted, and/or selected using one or more supervised and/or unsupervised machine-learning processes and/or models as described in further detail below. In some embodiments, training data 1104 may be classified using training data classifier 1116. Training data classifier 1116 may include a classifier. Training data classifier 1116 may utilize a mathematical model, neural net, or program generated by a machine learning algorithm. A machine learning algorithm of training data classifier 1116 may include a classification algorithm. A “classification algorithm” as used in this disclosure is one or more computer processes that generate a classifier from training data. A classification algorithm may sort inputs into categories and/or bins of data. A classification algorithm may output categories of data and/or labels associated with the data. A classifier may be configured to output a datum that labels or otherwise identifies a set of data that may be clustered together. Machine-learning module 1100 may generate a classifier, such as training data classifier 1116 using a classification algorithm. Classification may be performed using, without limitation, linear classifiers such as without limitation logistic regression and/or naive Bayes classifiers, nearest neighbor classifiers such ask-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 1116 may classify elements of training data to one or more faces.

Still referring to FIG. 11, machine-learning module 1100 may be configured to perform a lazy-learning process 1120 which may include a “lazy loading” or “call-when-needed” process and/or protocol. A “lazy-learning process” may include a process in which machine learning is performed upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 1104. Heuristic may include selecting some number of highest-ranking associations and/or training data 1104 elements. Lazy learning may implement any suitable lazy learning algorithm, including without limitation a K-nearest neighbors algorithm, a lazy naïve Bayes algorithm, or the like. Persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Still referring to FIG. 11, machine-learning processes as described in this disclosure may be used to generate machine-learning models 1124. A “machine-learning model” as used in this disclosure is a mathematical and/or algorithmic representation of a relationship between inputs and outputs, as generated using any machine-learning process including without limitation any process as described above, and stored in memory. For instance, an input may be sent to machine-learning model 1124, which once created, may generate an output as a function of a relationship that was derived. For instance, and without limitation, a linear regression model, generated using a linear regression algorithm, may compute a linear combination of input data using coefficients derived during machine-learning processes to calculate an output. As a further non-limiting example, machine-learning model 1124 may be generated by creating an artificial neural network, such as a convolutional neural network comprising an input layer of nodes, one or more intermediate layers, and an output layer of nodes. Connections between nodes may be created via the process of “training” the network, in which elements from a training data 1104 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 11, machine-learning algorithms may include supervised machine-learning process 1128. A “supervised machine learning process” as used in this disclosure is one or more algorithms that receive labelled input data and generate outputs according to the labelled input data. For instance, supervised machine learning process 1128 may include motion data as described above as inputs, symptoms of a movement disorder as outputs, and a scoring function representing a desired form of relationship to be detected between inputs and outputs. A scoring function may maximize a probability that a given input and/or combination of elements inputs is associated with a given output to minimize a probability that a given input is not associated with a given output. A scoring function may be expressed as a risk function representing an “expected loss” of an algorithm relating inputs to outputs, where loss is computed as an error function representing a degree to which a prediction generated by the relation is incorrect when compared to a given input-output pair provided in training data 1104. Persons skilled in the art, upon reviewing the entirety of this disclosure, will be aware of various possible variations of at least a supervised machine-learning process 1128 that may be used to determine relation between inputs and outputs. Supervised machine-learning processes may include classification algorithms as defined above.

Further referring to FIG. 11, machine learning processes may include unsupervised machine-learning processes 1132. An “unsupervised machine-learning process” as used in this disclosure is a process that calculates relationships in one or more datasets without labelled training data. Unsupervised machine-learning process 1132 may be free to discover any structure, relationship, and/or correlation provided in training data 1104. Unsupervised machine-learning process 1132 may not require a response variable. Unsupervised machine-learning process 1132 may calculate patterns, inferences, correlations, and the like between two or more variables of training data 1104. In some embodiments, unsupervised machine-learning process 1132 may determine a degree of correlation between two or more elements of training data 1104.

Still referring to FIG. 11, machine-learning module 1100 may be designed and configured to create a machine-learning model 1124 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm). Coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of I divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought. Similar methods to those described above may be applied to minimize error functions, as will be apparent to persons skilled in the art upon reviewing the entirety of this disclosure.

Continuing to refer to FIG. 11, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

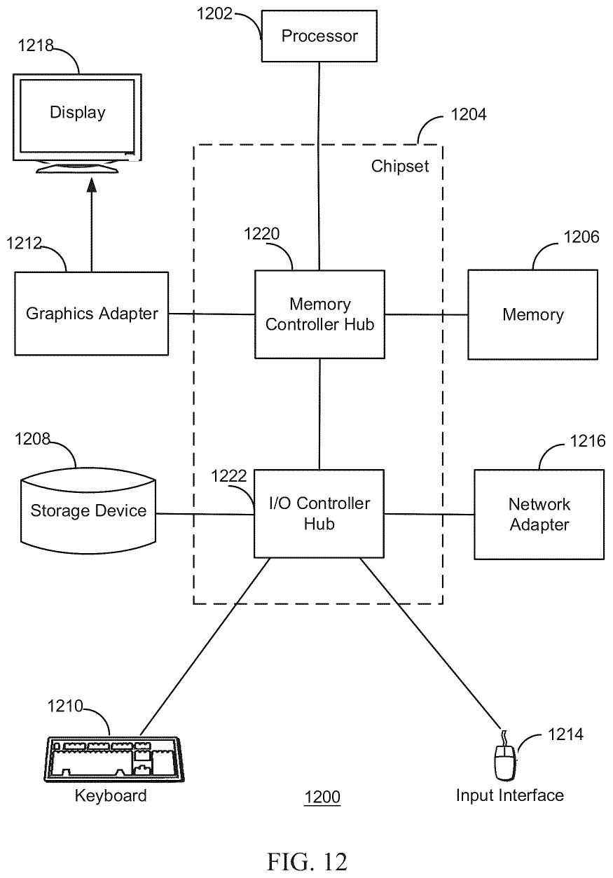

FIG. 12 illustrates an example computer for implementing the systems and methods as described herein. In some embodiments, the computing device includes at least one processor 1202 coupled to a chipset 1204. The chipset 1204 includes a memory controller hub 1220 and an input/output (I/O) controller hub 1222. A memory 1206 and a graphics adapter 1212 are coupled to the memory controller hub 1220, and a display 1218 is coupled to the graphics adapter 1212. A storage device 1208, an input interface 1214, and network adapter 1216 are coupled to the I/O controller hub 1222. Other embodiments of the computing device have different architectures.

The storage device 1208 is a non-transitory computer-readable storage medium such as a hard drive, compact disk read-only memory (CD-ROM), DVD, or a solid-state memory device. The memory 1206 holds instructions and data used by the processor 1202. The input interface 1214 is a touch-screen interface, a mouse, track ball, or other type of input interface, a keyboard, or some combination thereof, and is used to input data into the computing device. In some embodiments, the computing device may be configured to receive input (e.g., commands) from the input interface 1214 via gestures from the user. The graphics adapter 1212 displays images and other information on the display 1218. The network adapter 1216 couples the computing device to one or more computer networks.

The graphics adapter 1212 displays representations, graphs, tables, and other information on the display 1218. In various embodiments, the display 1218 is configured such that the user (e.g., data scientists, data owners, data partners) may input user selections on the display 1218. In one embodiment, the display 1218 may include a touch interface. In various embodiments, the display 1218 can show one or more predicted lead time for providing a customer order.

The computing device 1200 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 1208, loaded into the memory 1206, and executed by the processor 1202.

The types of computing devices 1200 can vary from the embodiments described herein. For example, a system can run in a single computer 1200 or multiple computers 1200 communicating with each other through a network such as in a server farm. In another example, the computing device 1200 can lack some of the components described above, such as graphics adapters 1212, input interface 1214, and displays 1218.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A wearable device for mitigating movement disorder symptoms of a subject, the device comprising:
a housing;
an attachment system coupled to the housing and configured to be attached to a body part of a subject;
a sensor disposed in the housing and configured to provide a sensor output related to an involuntary movement of the body part;
a processing unit disposed in the housing and operationally coupled to the sensor, wherein the processing unit is configured to generate a waveform output based on the sensor output; and
a noise filter configured to remove, from the sensor output, noise unrelated to the movement disorder symptoms, wherein the processing unit is further configured to:
extract a fundamental frequency from the sensor output through spectral analysis;
generate the waveform output by processing the sensor output based on the fundamental frequency; and
use a second filter to remove, from the waveform output, frequencies outside a specified range associated with the fundamental frequency; and
an electric transducer disposed in the attachment system and operationally coupled to the processing unit to provide the waveform output to the body part, wherein the processing unit is further configured to control electric transducer to deliver the waveform output to the body part of the subject.

2. The device of claim 1, wherein the electrical waveform output is applied to one or more mechanoreceptors of the body part of the subject.

3. The device of claim 1, wherein the processing unit is further configured to detect a freezing gait of a patient with Parkinson's Disease.

4. The device of claim 3, wherein the processing unit is further configured to control the electric transducer to relieve the freezing gait of a patient with Parkinson's Disease.

5. The device of claim 1, wherein the attachment system includes a wristband, and the electric transducer is disposed in a circumference of the wristband.

6. The device of claim 1, wherein the device is operated by a button on a face of the device, and the button is configured on the face to allow for ease of use by a patient whose fine motor control is affected by a neurological movement disorder.

7. The device of claim 1, wherein the spectral analysis includes applying a Fourier Transform to the sensor output.

8. The device of claim 1, wherein the processing unit is further configured to extract temporal features from the sensor output.

9. The device of claim 1, wherein the processing unit is further configured to operate in a first mode, wherein in the first mode the processing unit passively detects a movement disorder symptom above a threshold value.

10. The device of claim 1, wherein the processing unit is further configured to: receive a selection of a specific waveform parameter from a predefined list of waveforms; and apply the specific waveform parameter to the body part of the user based on the selection.

11. A method of mitigating a movement disorder of a user using a wearable device, comprising:
generating, by a sensor of the wearable device, sensor output;
filtering, through a filter, noise unrelated to a movement disorder symptom from the sensor output;
extracting, by a processing unit of the wearable device, a fundamental frequency from the sensor output through spectral analysis;
generating, by the processing unit, a waveform output based on the fundamental frequency; and
applying, by an electric transducer disposed in an attachment system of the wearable device, the waveform output to a body part of a subject.

12. The method of claim 11, further comprising applying the waveform output to mechanoreceptors of the body part of the subject.

13. The method of claim 11, wherein extracting the fundamental frequency through spectral analysis includes applying a Fourier transform to the sensor output.

14. The method of claim 11, further comprising extracting, by the processing unit, temporal features of the sensor output.

15. The method of claim 11, further comprising operating, by the processing unit, in a first mode, wherein operating in a first mode comprises passively detecting a movement disorder symptom of the subject above a threshold value.

16. The method of claim 11, further comprising detecting, by the processing unit, a freezing gait of the subject.

17. The method of claim 16, further comprising controlling, by the processing unit, the electric transducer to relieve the freezing gait of a patient with Parkinson's Disease.

18. The method of claim 11, wherein the attachment system includes a wristband, and the electric transducer is disposed in a circumference of the wristband.

19. The method of claim 11, wherein the wearable device is operated by a button on a face of the device, and the button is configured on the face to allow for ease of use by a patient whose fine motor control is affected by a neurological movement disorder.

20. The method of claim 11, further comprising generating and applying a train of waveform outputs to the body part of the subject.
